(19) United States
(12) Patent Application Publication
(10) Pub. No.: US 2017/0326375 A1
(43) Pub. Date: Nov. 16, 2017

(54) SYSTEM AND METHOD FOR STORING AND RETRIEVING DATA FROM NEUROSTIMULATION SYSTEMS

(71) Applicant: BOSTON SCIENTIFIC NEUROMODULATION CORPORATION, Valencia, CA (US)

(72) Inventors: MATTHEW L. MCDONALD, PASADENA, CA (US); SAMUEL TAHMASIAN, GLENDALE, CA (US); JILLIAN DOUBEK, LOS ANGELES, CA (US)

(73) Assignee: BOSTON SCIENTIFIC NEUROMODULATION CORPORATION, Valencia, CA (US)

(21) Appl. No.: 15/595,367

(22) Filed: May 15, 2017

Related U.S. Application Data
(60) Provisional application No. 62/337,024, filed on May 16, 2016.

(51) Int. Cl. A61N 1/372; A61N 1/375; A61N 1/378; A61N 1/36

(52) U.S. Cl. CPC A61N 1/37247; A61N 1/3787; A61N 1/3605; A61N 1/37252; A61N 1/3756

(57) ABSTRACT

Implantable medical device system configured to use removable memory media or wireless communication to avoid having to store charging and other data on the implantable device itself. Some examples include systems of external devices such as an external charger, external patient controller, and/or clinician programmer, that communicate with one another the details of a given implantable device operation using removable memory such as a USB memory drive.

### CROSS REFERENCE TO RELATED APPLICATIONS

The present application claims the benefit of and priority to U.S. Provisional Patent Application Ser. No. 62/337,024, filed on May 16, 2016 and titled SYSTEM AND METHOD FOR STORING AND RETRIEVING DATA FROM NEUROSTIMULATION SYSTEMS, the disclosure of which is incorporated herein by reference.

### BACKGROUND

Implantable and/or wearable stimulations systems for the treatment of various diseases and disorders of the neurological system have proven effective in a wide variety of ways. For example, spinal cord stimulation (SCS) systems are accepted treatments for chronic pain syndromes. Deep brain stimulation (DBS) may be used for various purposes, and are gaining acceptance as well including for treatment of movement and tremor disorders. Peripheral nerve stimulation (PNS) systems have also been shown effective for certain indications, and functional electrical stimulation (FES) has been investigated for restoration of functionality to paralyzed extremities. These and other therapies are under investigation for numerous indications beyond those already in use.

Historically many of the available implantable systems included a relatively bulky implantable pulse generator attached to a lead. The pulse generator may have had a volume on the order of twenty cubic centimeters or more for rechargeable systems, and thirty cubic centimeters or more for non-rechargeable or “primary cell” systems. Much smaller implants are under development, having volumes in the range of less than about five cubic centimeters, which may omit the use of a separate lead. Some examples are discussed, for example, in U.S. Pat. Nos. 8,630,705 and 7,437,193. The small size of such systems creates potential constraints on implantable device memory and power usage. New and alternative solutions to potential memory constraints are desired.

### Overview

A first non-limiting example takes the form of a charging apparatus for use with an implantable medical device, the charging apparatus comprising: a charging circuit for generating an output magnetic and/or electrical field for charging an implantable medical device; a monitoring module for determining and storing charging data related to the operation of the charging circuit; and an output circuit for offloading data from the monitoring module to at least one of a removable memory and/or a separate device connectible wirelessly to the output circuit.

A second non-limiting example takes the form of a charging apparatus as in the first non-limiting example, wherein the monitoring module determines the charging data including at least one of duration of charging events or frequency of charging events without using charge duration or frequency data stored by the implantable medical device. A third non-limiting example takes the form of a charging apparatus as in the first non-limiting example, wherein the monitoring module is configured to send data from the monitoring module over the internet to a central repository. A fourth non-limiting example takes the form of a charging apparatus as in the first non-limiting example, wherein the monitoring module stores charging records for each of a plurality of charging sessions in which the charging apparatus is used comprising: an identifier for a specific implantable medical device charged in a charging session; an identifier of when the charging session with the specific implantable medical device occurred; and at least one parameter of the charging session with the specific implantable medical device. A fifth non-limiting example takes the form of a charging apparatus as in the fourth non-limiting example, wherein the at least one parameter includes a measure of charging efficiency obtained by communication with an implantable medical device while charging the implantable medical device.

A sixth non-limiting example takes the form of an implantable medical device patient system comprising: a charging apparatus as in any of the first to fifth non-limiting examples; and an external control device configured for communication with the implantable medical device and controlling therapy outputs of the implantable medical device; wherein: the output circuit of the charging apparatus is configured to load charging data on a removable memory device; and the external control device is configured to read and/or display charging data from the removable memory device.

A seventh non-limiting example takes the form of a system as in the sixth non-limiting example, wherein the external control device takes the form of a patient remote control or a clinician programmer and the external control device comprises a memory to store therapy usage data for the implantable medical device, a correlation module to correlate charging data from the charging apparatus to the therapy usage data and a device analysis module to determine characteristics of the operation of the implantable medical device from the charging data and the therapy usage data.

An eighth non-limiting example takes the form of a system as in the sixth non-limiting example, wherein the characteristics of the operation of the implantable medical device comprise an indication of the status of a rechargeable power supply of the implantable medical device. A ninth non-limiting example takes the form of a system as in the sixth non-limiting example, wherein the characteristics of the operation of the implantable medical device comprise an indication of the efficiency of charging of the implantable medical device.

A tenth non-limiting example takes the form of an implantable medical device patient system comprising: a charging apparatus as in any of the first to fifth non-limiting examples; and a patient remote control configured for communication with the implantable medical device and controlling therapy outputs of the implantable medical device; wherein: the output circuit of the charging apparatus is configured to communicate with the patient remote control to offload the charging data from the monitoring module; and the patient remote control comprises a memory to store therapy usage data for the implantable medical device, a correlation module to correlate charging data from the charging apparatus to the therapy usage data and a device analysis module to determine characteristics of the operation of the implantable medical device from the charging data and the therapy usage data.

An eleventh non-limiting example takes the form of a system as in the tenth non-limiting example, wherein the characteristics of the operation of the implantable medical device comprise an indication of the status of a rechargeable power supply of the implantable medical device. A twelfth non-limiting example takes the form of a system as in the tenth non-limiting example, wherein the characteristics of the operation of the implantable medical device comprise an indication of the efficiency of therapy delivery of the implantable medical device.

A thirteenth non-limiting example takes the form of a system as in any of the sixth to twelfth non-limiting examples, further comprising a first implantable medical device having in the range of about two to about thirty-two electrical contacts thereon and a rechargeable power supply and therapy circuit therein for delivering therapy outputs via the electrical contacts. A fourteenth non-limiting example takes the form of a system as in the thirteenth non-limiting example, further comprising at least a second implantable medical devices each having a rechargeable power supply, wherein the charging data comprises records for each of the first and second medical devices.

A fifteenth non-limiting example takes the form of an implantable medical device system comprising: at least one implantable medical device having about two to about thirty-two electrodes thereon for therapy delivery, therapy circuitry for providing therapy via the electrodes, a rechargeable power source and charging circuitry for charging the rechargeable power source using power received from a charger apparatus; a charger apparatus comprising: a charging circuit for generating an output magnetic and/or electrical field for charging an implantable medical device; a monitoring module for determining and storing charging data related to the operation of the charging circuit; and an output circuit for offloading charging data from the monitoring module to a removable memory; an external control device configured to communicate with the implantable medical device and control therapy outputs from the implantable medical device, the external control device having a memory to store therapy usage data for the implantable medical device, and having a correlation module for obtaining charging data from the removable memory and correlating the charging data to therapy usage data to generate an output file containing correlated charging and therapy information.

A sixteenth non-limiting example takes the form of a method comprising: a charger as in any of the first to fifteenth non-limiting examples outputting energy to transcutaneously charge an implantable medical device in a charging session; the charger storing data relating to the charging session on a removable memory; and removing the removable memory and placing it into one of a patient remote control or a clinician programmer to obtain charge data. Additionally or alternatively, the sixteenth example may further include the patient remote control assimilating the charge data with therapy or other data gathered by the patient remote control. Additionally or alternatively, the sixteenth example may further include the physician programmer presenting information, analysis, or metrics from one or both of the charger or patient remote control to a physician after reading data from the removable memory.

This overview is intended to briefly introduce the subject matter of the present patent application, and is not intended to provide an exclusive or exhaustive explanation of the invention. The detailed description is included to provide further information.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative implanted system;

FIG. 2 is a block diagram of an illustrative implantable neurostimulator;

FIG. 3 is a block diagram of an illustrative charger for recharging a stimulator;

FIG. 4 is a block diagram of a patient remote control for controlling operation of a stimulator;

FIG. 5 is a block diagram of a clinician programmer for monitoring status of and controlling operation of a stimulator;

FIG. 6 shows an illustrative example at a system level;

FIG. 7 shows another illustrative example at a system level, this time with multiple implanted devices;

FIG. 8 shows another illustrative example at a system level, this time using an internet based solution;

FIG. 9 shows an illustrative charger with an implantable pulse generator and a removable memory;

FIG. 10 shows an illustrative patient remote control with an implantable pulse generator and a removeable memory;

FIG. 11 shows a data structure of an illustrative charge record;

FIG. 12 shows a data structure of an illustrative device record;

FIGS. 13-15 show illustrative methods in block flow form.

### DETAILED DESCRIPTION

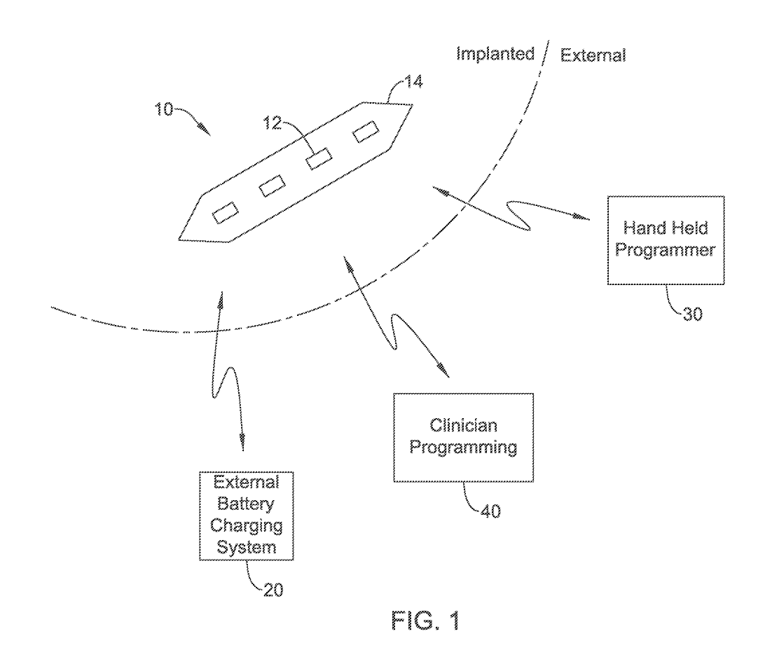

FIG. 1 shows an illustrative implanted system. A miniaturized implantable pulse generator (IPG) is shown at 10, and includes a plurality of electrodes 12 on a housing 14. The electrodes 12 may be formed of a conductive material, such as a conductive metal like titanium, stainless steel or numerous other examples, or a conductive polymer. The housing 14 may hermetically seal the device 10 to protect internal power supply and electronics that control operations of the device 10, including for example, delivering therapy via the electrodes 12. In some examples, one or more components such as the batteries may be exposed on one or more surface to the external environment, with the housing 14 hermetically sealing other componentry as needed. Any suitable number of electrodes may be provided, on the housing 14, with a likely range of 1 to about 16. The housing, aside from the electrodes 12, may serve as a return or indifferent electrode for therapy purposes in some examples. While several embodiments may entirely omit a lead in association with device 10, other embodiments may include a removable or permanently attached lead with one or more additional electrodes.

The IPG 10 is shown in an implanted state, with the dot-dot-dash line representing the patient's skin. The IPG may be placed near a suitable neurological structure to provide electrical therapy using an output voltage or current, for example, to affect nearby neurological function and achieve a desirable therapeutic effect. For example, the IPG may be placed inside the cranium, in or near the spinal column, or in the vicinity of a targeted nerve such as the vagus nerve, a peripheral nerve, or any other suitable location.

Various externals can be used with the IPG 10 in this example. An external charger 20 may be used to recharge and communicate with the IPG 10. A hand-held programmer or patient remote control 30 may be used by the patient to control therapy operations of the IPG 10. A clinician programmer 40 may be used by a clinician to set up the IPG 10 with therapy patterns and to define programs that can be operated by the IPG 10 when operated using the patient remote control 30. The clinician programmer 40 and/or patient remote control 30 can also be used to perform diagnostic inquiries on the IPG 10. Various inductive and/or RF links are illustrated in the system

In the illustrative example, the IPG includes a replenishable power supply, for example, a rechargeable lithium ion battery (or other chemistry), or a capacitor such as a supercapacitor. Recharging energy may be provided by the charger using, for example, a varying magnetic field generated using an inductive coil. For example, the IPG may include a coil that is responsive to an applied magnetic field from the external charger 20. Other links (RF, optical, thermal, sonic, etc.) may be used instead. A non-rechargeable or “primary cell” system may be used instead. Some investigation has been performed into biologic power supplies or power supplies that take advantage of patient bodily action (such as movement); such alternative power supply may be used instead of or in addition to a charger/coil using magnetic or electric fields.

Traditional approaches to the use of an implantable neuromodulation system having a canister attached to a lead would use the implanted device as the hub for information about the implanted system. Therefore a traditional implanted device would maintain histories of charging session data (including for example, duration of charging sessions and frequency at which such sessions occur) as well as histories of therapy session data (electrode selection, amplitude of therapy, how often therapy is used, and for how long a therapy session lasts, for example). Charge session data can be used to assess the operation of the rechargeable circuit in the implanted device for example; for example longer and more frequent charge session may suggest a battery that is nearing end of life, a malfunction in the device, or possibly movement of the implanted device causing a reduction in efficiency of charging. Therapy data can be used to optimize device settings and to assess a patient's progress in dealing with a particular disease state. Additionally, therapy data may be useful to predict device longevity by suggesting how frequently recharging will be needed.

More traditional implantable neurostimulation devices typically have ample space and power capacity to allow for data storage in volatile and/or non-volatile memory, with sizes in the range of twenty to thirty-five cubic centimeters, for example. For the system shown in FIG. 1, the idea is to have IPG 10 with a volume of under ten cubic centimeters, and more preferably in the range of about one to about five cubic centimeters. The smaller volume not only means less available space for components such as memory, it also places a premium on the use of power to perform memory related functions such as writing data to memory and maintaining volatile memory to avoid data loss. Approaches that allow the implanted device to play a less prominent role for keeping charging and therapy data are desired.

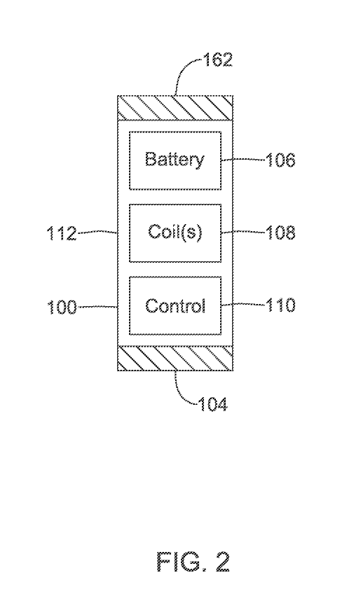

FIG. 2 is a block diagram of an IPG which may take the form of a rechargeable and implantable neurostimulator. The IPG 100 is shown with first and second end electrodes 102, 104; other configurations and number of electrodes may be used instead. A battery 106 is provided as the power source, and is coupled to the coil(s) 108 to allow for recharging for example by inductive power transfer. A control circuit is provided as shown at 110 and may include any suitable analog or digital operational circuitry including, for example, such logic, amplifiers, impedance circuits, application-specific integrated circuits (ASIC), microprocessors or microcontrollers, and memory, including volatile and/or non-volatile memory as may be needed to perform the functions needed or such a system. Some examples of implantable device circuitry are shown, for example, in U.S. Pat. Nos. 9,242,106 and 9,072,904, the disclosures of which are incorporated herein by reference. The device circuitry that is part of the control block 110 may include circuitry for controlling the battery recharging that is done via the coils 108, as well as circuitry dedicated to telemetry using the coil(s) 108, and circuitry dedicated to controlling electrical therapeutic outputs via the electrodes 102, 104.

There may be one or plural coils 108; for example one coil may be sized, placed and coupled to circuitry for use as a charging coil for recharging the battery, while another (typically smaller) coil is sized, placed, and coupled to circuitry for use as a telemetry coil. For example, the charging coil may be sized for inductive telemetry, while the telemetry coil may in fact serve as an antenna for radiofrequency (RF) communication in the ISM or Medradio bands. No particular shape should be inferred form the use of the term “coil” in this context. In an alternative example, a single coil may be used for both telemetry and charging.

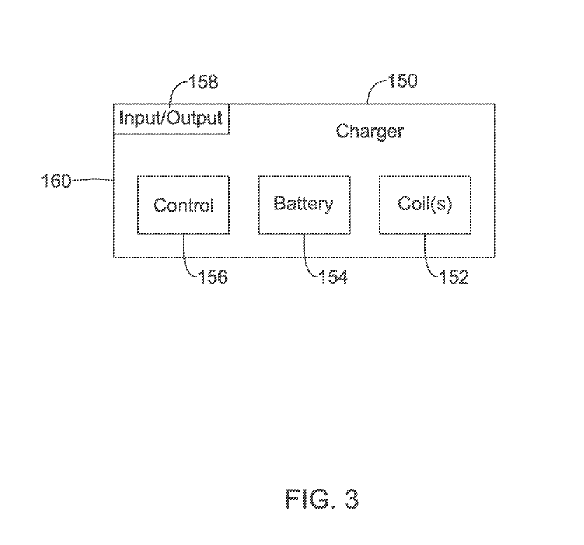

FIG. 3 is a block diagram of an illustrative charger for recharging a stimulator. Charger 150 includes one or plural coils 152 corresponding in part to the coil(s) of the IPG. Commonly such chargers include a battery 154 as power supply, which may be rechargeable. For example, a charger 150 may be adapted for use with a recharging cradle or an adapter, either of which may operate using line voltage by plugging into an outlet. A control circuit 156 is provided, as well as an input/output circuit. The control circuit 156 may control the operation of the charging and/or telemetry coils to prevent overheating and to selectively optimize charging efficiency by, for example, adjusting frequency and/or impedance matching. The input/output may include, for example, a user interface having buttons, lights and/or a screen to provide information to the user regarding the status of charging operations, the charger battery 154 status, and/or information relating to whether the charger 150 is appropriately aligned with an IPG to achieve efficient charging, as is known in the art.

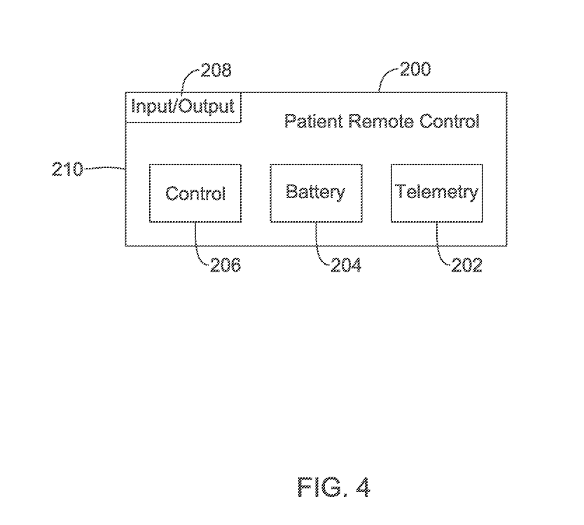

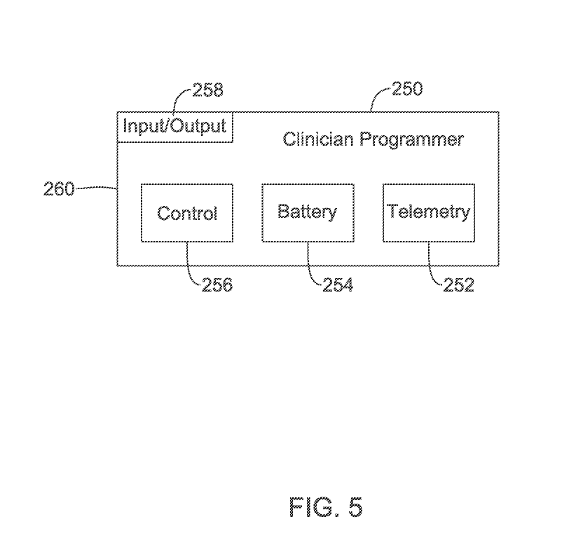

In addition, the input/output 158 may be configured to couple with a removable memory element as further described below. Input/output may also be configured to generate a wireless signal using, for example Bluetooth, WiFi, and/or other known wireless protocol to allow linking to a router or computing device, or to another device of the implantable medical device system such as the patient remote control (FIG. 4) or clinician programmer (FIG. 5).

The charger may be provided in a single housing 160 or may include first and second linked together housings having, for example, the charging and/or telemetry coils on a wand or disc tethered to a main housing. Such tethering together may be provided to allow, for example, the patient to place the charging coil directly over a device implanted on the patient's back, while holding the rest of the charger in his or her hand, to make control easy.

FIG. 4 is a block diagram of a patient remote control for controlling operation of a stimulator. The patient remote control 200 may include a telemetry circuit 202 for communicating with an IPG, a battery 204, which again may be rechargeable using a plug-in adaptor or cradle, as desired. A control circuit 206 is provided to control operation of the device. An input/output 208 block may include a screen or other user interface and controls to allow the patient to use the patient remote control to activate and otherwise control and, in some instances, modify therapy. For example, the patient may be allowed to shift the locus of therapy by selecting and deselecting electrodes to move therapy up or down or back and forth relative to an electrode array, or the patient may be allowed to increase or decrease therapy amplitude via buttons or a touchscreen provided in the input/output 208. As before, the patient remote control 200 may include a single housing 210 or may have multiple housings to allow the telemetry coil to be placed near an implanted device while the rest of the unit is easily accessible to the user.

In addition, the patient remote control 200 comprises an input/output 208 that may be configured to couple with a removable memory element as further described below. Input/output may also be configured to generate a wireless signal using, for example Bluetooth, WiFi, and/or other accepted wireless protocols such as Zigbee to allow linking to a router and hence to the Internet, or to a computing device, or to another device of the implantable medical device system such as the charger (FIG. 3) or clinician programmer (FIG. 5).

FIG. 5 is a block diagram of a clinician programmer for monitoring status of and controlling operation of a stimulator. The clinician programmer 250 may include a telemetry circuit 252, battery power 254 and control circuitry 256. The clinician programmer 250 will typically have significant memory and computing resources similar to modern laptop or tablet computers (as may the patient remote control, which can be a commercial, off-the-shelf component such as a smartphone or tablet computer, having specialized software and/or hardware extensions to allow use as a medical device). In some examples, the patient programmer and clinician programmer may be one and the same.

The clinician programmer 250 can be used to set up and program the IPG. For example, the clinician programmer 250 may determine and load therapeutic programs onto the IPG and set boundaries around the changes that may be made by the patient remote control. A therapeutic program may designate, for example, a selection of electrodes to be used when therapy is delivered, in pairs or other combinations, designating anode/cathode arrangements and the type of therapy (such current or voltage controlled), and the shape, amplitude, duration and frequency of therapy stimuli. For example, a relatively simple program may deliver biphasic square waves of 8 milliseconds duration with 1 millisecond interphase delay, delivered at 20 Hertz (that is, once every 50 milliseconds) with 1 mA current-controlled output amplitude between a selected pair of electrodes. Any desired/suitable therapy may be delivered. For example, low frequency, tonic, burst, high frequency, and/or non-square wave therapy outputs maybe generated and programmed, taking into consideration any hardware limitations of the IPG. Technology and therapy may progress to more complex therapy outputs as desired.

In addition, the clinician programmer 250 comprises an input/output that may be configured to couple with a removable memory element as further described below. Input/output may also be configured to generate a wireless signal using, for example Bluetooth, WiFi, and/or other accepted wireless protocols such as Zigbee to allow linking to a router and hence to the Internet, or to a computing device, or to another device of the implantable medical device system such as the charger (FIG. 3) or patient remote control (FIG. 4).

As before, the clinician programmer 250 may be enclosed in a single housing, or, in some examples, may provide for the telemetry circuitry 252 to include a wand that can be placed over a patient device on the skin of the patient.

In some examples, the external devices of FIGS. 3-5 may operate in conjunction with a removable memory that can be used to transfer information therebetween. In other examples, communication between the external devices in FIGS. 3-5 may take place using methods noted above such as Wifi, Zigbee, and Bluetooth protocols, as well as other methods such as infrared communication, other wireless methods, and/or via wired connection.

Communication between the IPG (FIG. 2) and any of the charger (FIG. 3), patient remote control (FIG. 4), and/or clinician programmer (FIG. 5) may also take the form of conducted communication in which messages may be encoded in outputs delivered via the electrodes 102, 104 of the IPG. For conducted communication, cutaneous electrodes may be placed directly on the skin of the patient to achieve desired connectivity. Conducted communication may be provided instead of or as an option in addition to any of RF or inductive communication.

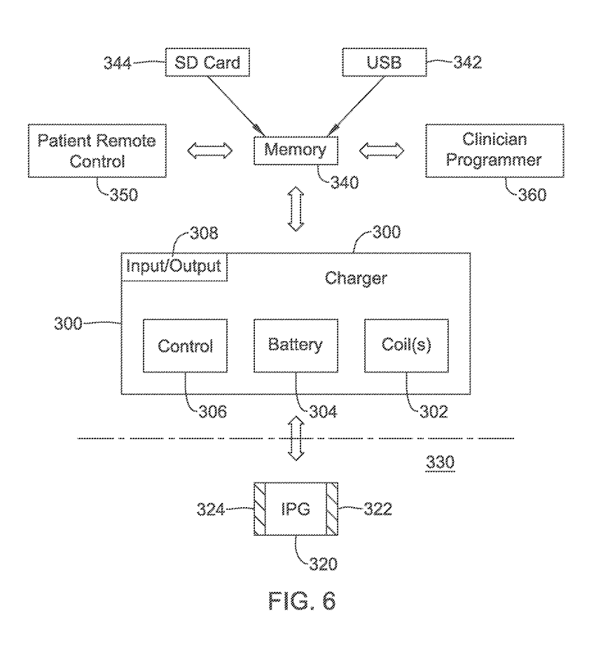

FIG. 6 shows an illustrative example at a system level. A charger 300 is shown including, for example, coils 302, battery 304 and control circuitry 306, as well as input/output 308 circuitry and/or ports. The charger 300 is operable to provide energy in a charging session to an IPG 320 having electrodes 322, 324. The energy transfer may be, for example, by inductive coupling or other method. The IPG 320 is shown as being implanted in patient 330.

In this example, the IPG 320 has limited power and memory capabilities. Therefore the charger 300 records data related to the charging session. Such data may include, for example, identifying information for the IPG 320, indications of the duration of the charging, the time since last charging, and the status of the battery of the IPG 320 at the beginning and end of the charging session (indicating for example the depth of discharge, such as, 75% discharged at start of the session, and 0% discharged at the end of the session). Additional measurables may be recorded as well including, for example, indications of the coupling efficiency which may be measured by the charger by monitoring the reflected impedance of the IPG 320, or by obtaining data from the IPG 320 indicating instantaneous or average current obtained from the recharge circuitry. Other measures may be obtained as well. Some data may be generated by the charger 300, while other data may be obtained by communication with the IPG 320.

In the prior art, charging session data would ordinarily have been stored by the IPG and provided to a clinician programmer 360 directly during a later programming session. For example, the clinician programmer 360 would be used to allow a physician to review charging session data obtained directly from the IPG.

In contrast to such prior art, in the example of FIG. 6, the IPG 320 may store only minimal, or even no charging session data. In one example, the IPG 320 stores simply a timestamp of the most recent charging session; or a timestamp plus an indication of battery status at the end of a most recent charging session. Other data related to the session itself (duration, efficiency, begin and end depth of discharge, time since prior charge) would only be held by the charger 300.

In this example the charger 300 then loads charging session data on a removable memory 340. In some examples the removable memory 340 is a universal serial bus (USB) memory stick or “thumb drive”. In other examples, the removable memory 340 may take the form of a secure digital (SD) card. In yet other examples, other removable media may be used including, for example, a CD or DVD. The removable memory 340 can then be used to take the charging session data to the patient remote control 350 and/or the clinician programmer 360.

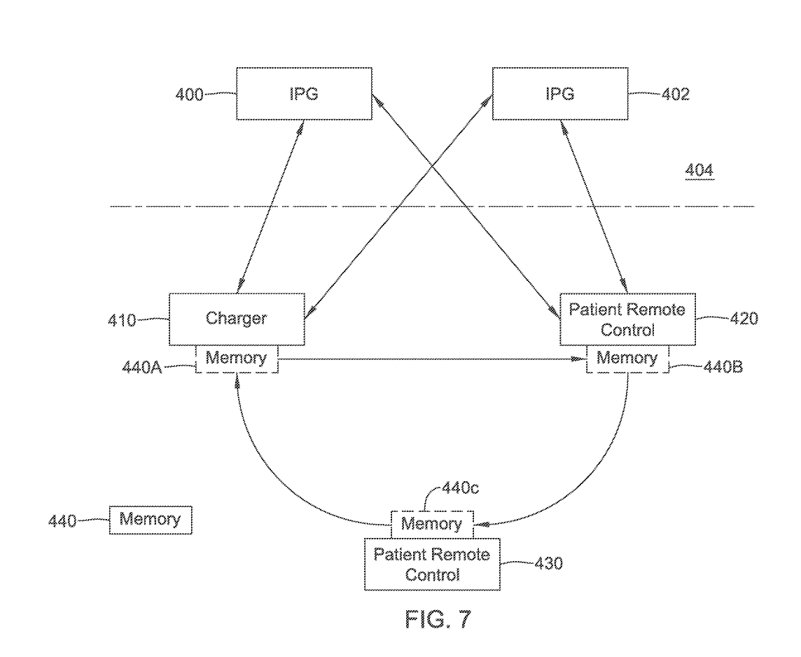

FIG. 7 shows another illustrative example at a system level, this time with multiple implanted devices and illustrating a sequence of actions with the removable memory. In this example, a first IPG 400 and a second IPG 402 are shown implanted in patient 404. A charger 410 and patient remote control 420 are configured to interact with each of the IPGs 400, 402. For example, the charger 410 would be placed over IPG 400 to charge it, and would subsequently be placed over IPG 402 to charge it as well. It may be possible to charge both IPGs 400, 402 at the same time depending on proximity and field strength, among other factors. In another example, only one of the IPGs 400, 402 is in the patient 404.

Regardless how many IPGs 400, 402 there are, the illustrative example includes a removable memory 440 which may be, for example, a USB memory drive, SD card or other media. The memory 440 can be attached and detached to several external devices during use. In the example of FIG. 7, the memory 440 can be coupled to the charger 410 during a charging session, as indicated at 440A. The memory 440 can also be coupled to the patient remote control 420 during a therapy session, as indicated at 440B. The memory 440 can later be coupled to the clinician programmer 430 such that a physician can obtain records of charging and therapy sessions for review.

In some examples, the charger 410 and patient remote control 420 create independent records for storage on the memory 440. In other examples, one of the charger 410 or patient remote control 420 may construct status files relating to device and/or system status and including one or more of: charging session data, therapy session data, and analytics based on combinations of the charging session data and therapy session data, or other inputs such as patient derived inputs. For example, patient derived inputs may include answers to questions related to activity, mobility, or pain, which may be administered via a user interface of one of the charger 410 and/or patient remote control 420.

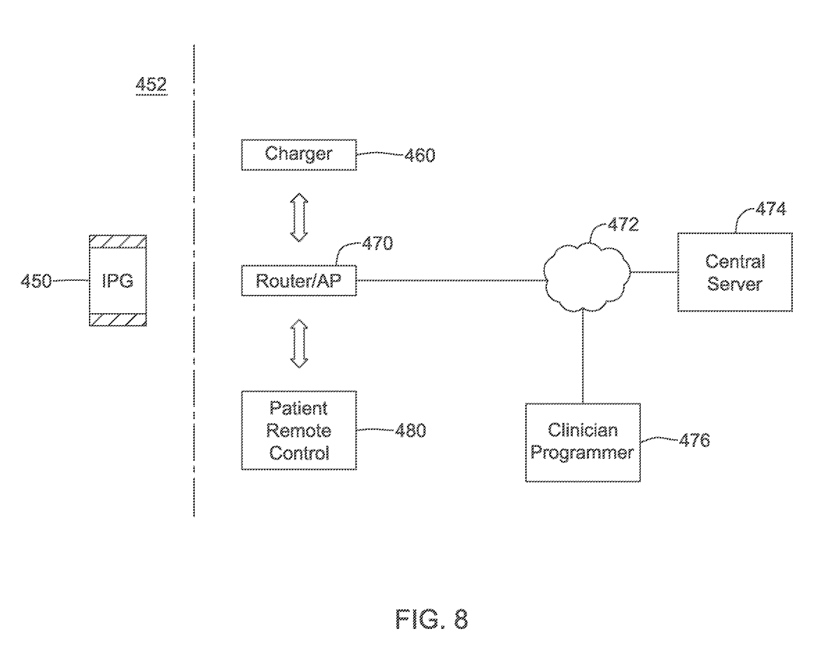

FIG. 8 shows another illustrative example at a system level, this time using an internet based solution. In this illustration, the IPG 450 is implanted in patient 452. A charger 460 and patient remote control 480 are shown as well and may each interact with the IPG 450 as with other examples. A router or access point 470 is also illustrated. The charger 460 and patient remote control 480 may communicate to the router via, for example, WiFi, or other wireless or wired communication, in real time or by periodic or occasional upload of data to the cloud/internet 472, where it may be directed to a central server 474. The central server may be administered by the manufacturer of the IPG 450, for example, or by a third party administrator, a clinic, or a hospital system. The clinician programmer 476 would be able to use internet connectivity to access patient records on the central server 474 when the patient comes into the clinic, avoiding a need for the IPG 450 to store such data using its limited capacity.

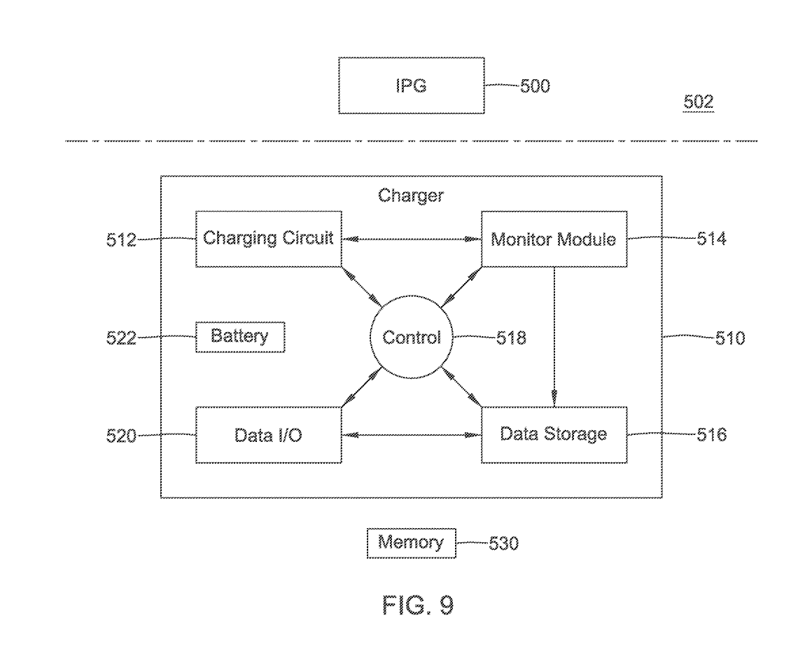

FIG. 9 shows an illustrative charger with an implantable pulse generator and a removable memory. The IPG 500 may be implanted in patient 502. The charger 510 is shown as comprising a number of functional blocks including a charging circuit 512, a monitoring module 514, a data storage 516, a control block 518, data input/output block 520, and a battery 522. Illustratively, the monitoring module 514 is used to gather and accumulate data related to a charging session, which may ultimately be stored in the data storage block 516 by the control block 518 until such data may be off-loaded via the data input/output block 520 by, for example, writing to a removable memory device 530 that may be, for example, an SD card or USB device (FIGS. 6-7), or by transmission via WiFi or wired connection (FIG. 8) to a server.

Separate blocks and various internal connections are shown for illustration, however, it should be understood that the individual blocks 512 to 522 need not be physically separate modules. For example, the charging circuit 512 may include a charger coil adapted to generate a magnetic field for output and use in charging. Various known circuits may be used to power the charging circuit 512 from the battery 522 to provide an output field of a desired frequency and power level. The charging circuit 512 and/or control block 518 may include safety circuitry to monitor and modulate, for example, total current and temperature, as both may rise during operation. Data obtained from safety circuitry may be included in the charging session data. The battery 522 may use any suitable battery chemistry; in some examples, a rechargeable lithium battery 522 may be used. The control block 518 may include, for example, a microprocessor or microcontroller, as well as suitable analog and digital control circuitry and logic circuits. The data storage for the charger 510 may use any suitable storage structures including volatile and non-volatile memory.

In some examples, a separately provided charging circuit 512 is present, and obtains power from the battery 522 and control signals from the control block 518. For such an example, the monitoring module 514 may be distributed within the charging circuit 512 and control block 518. The monitoring module 514 may comprise, for example, current, voltage or field monitoring elements integrated into the charging circuit 512 and controlled via the control module 518 to generate information about charging. For example, reflected impedance may be monitored in the charging circuit by observing a voltage (or changes thereof) across the charging coil during charging; as the monitored voltage changes, it may be determined how well aligned the charger inductive coil is with respect to the charging coil of the IPG 500; such impedance informs an understanding of the efficiency of the charging cycle. Such impedance may also be used by the charger 510 to alert the user as to the alignment or misalignment between the IPG 500 and the charger 510, with the user instructed to reposition the charger 510 relative to the IPG 500 to achieve better alignment and faster, more efficient charging. Various suitable methods to establish the quality of alignment can be used and are known in the art.

During charging, the IPG 500 may issue communications regarding device usage and/or status to the charger 510, if desired. For example, the IPG 500 may indicate any existing errors or other flags, communication session data, status over time, cumulative current usage since the last charge session, stimulation parameters, stimulation usage, and/or information about any therapy delivered during charging or other device behavior, and/or any additional information gathered over time depending on device capability (such as device accelerometer data that may allow assessment of movement disorder or tremors, for example). Such information may be captured in the charge session data.

Thus the charging session data may include one or several of, for example, the depth of discharge of the IPG battery at the start of a session, the date/timestamp of the session, the duration of a session, the depth of discharge of the IPG battery at the end of a session, safety data, any reported diagnostic data from the IPG, data relating to the efficiency of charging, alignment of the IPG 500 and charger 510 during charting, and/or other information.

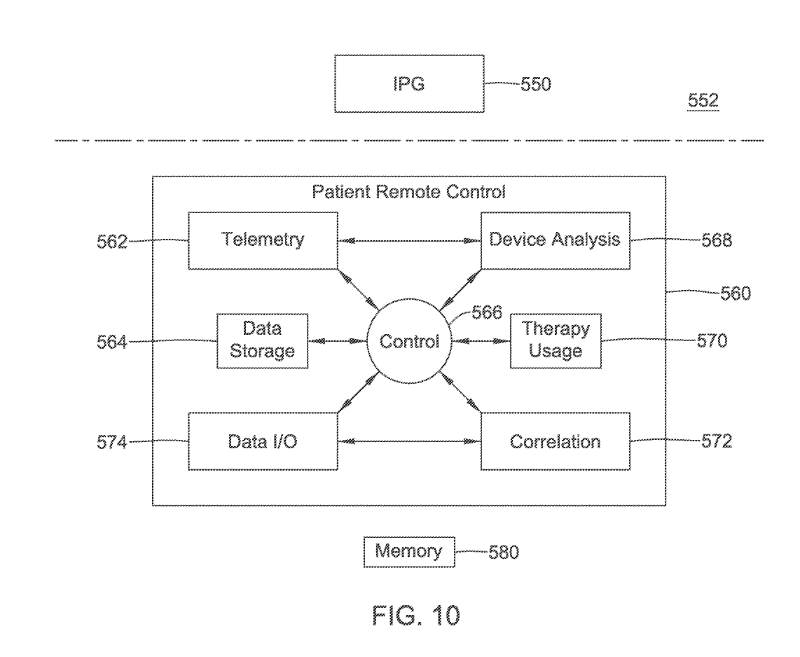

FIG. 10 shows an illustrative patient remote control with an implantable pulse generator and a removable memory. In this example, the IPG 550 may be implanted in the patient 552, and a patient remote control 560 can be used to control therapy delivery by the IPG 550. The patient remote control 560 is shown with a number of functional blocks therein. It should be understood that while the individual functional blocks may actually be physically separate circuits and elements, such as on plural hybrid circuit boards, in most examples at least some of the functional block may refer to circuit elements as well as software instructions, with the circuit elements distributed within the device and the software instructions be accessible and executable by the control block 566.

In the example shown, the patient remote control 560 includes functional blocks or sub-circuits for telemetry 564, memory 564, control block 566, device analysis 568, therapy usage 570, correlation 572, and data input/output. In an illustration, the telemetry circuit 562 may comprise controls for a telemetry output using, for example, RF or inductive communication including any necessary amplifiers, mixers, oscillators, crystals, and antennae or coils for such usage and may use, for example, a Medradio or ISM frequency band, or other frequency ranges or transmission types. As noted above, conducted communication may be used as an alternative to RF or inductive telemetry.

The telemetry circuit 562 serves the purpose of allowing data transfer with the IPG 550 including, for example, providing commands to the IPG 550 to deliver therapy, perform diagnostic tests, or provide information such as the IPG 550 identifier, battery status, any existing error or other flags, etc. The control block 566 can distribute obtained information for use in device analysis 568. Device analysis may include, for example, software instructions for determining and accumulating therapy information of the device and/or to review different data points provided by the IPG 550. For example, knowing the depth of battery discharge for the IPG both at a current time and at a prior time, such as during a prior interrogation by the patient remote control 560 or after a previous charging session, as well as how much therapy has been delivered, characteristics such as quiescent current or battery self-discharge may be assessed, as well as how efficiently therapy is being delivered.

The therapy usage block 570 may be used to track the commanded therapy delivered by the IPG 550. In some examples, the IPG may deliver therapy on its own, acting autonomously according to a stored program, with the patient remote control 560 used to modify therapy delivery. In other examples, the IPG 550 may only deliver therapy when commanded by the patient remote control. Data on autonomous therapy delivered may be obtained by the patient remote control 560 using telemetry 562 and passed by control block 566 to the therapy usage block 570. Combining device analysis 568 with therapy usage block 570, additional information may be gleaned and calculated as well, for example, knowing how much current discharge is reported by the battery during a given period of time while therapy is delivered allows an estimate to be made of the output impedance of therapy.

The data input/output block 574 may be used to retrieve and/or store data on a removable memory 530. Alternatively, the input/output block 574 may obtain or upload data via the Internet from a central server. In some examples, the data input/output block 574 is simply used to store data such as that obtained by the device analysis block 568 and/or therapy usage block 570. In other examples, the patient remote control is configured to obtain charging session data from the memory 530.

A correlation block 572 is also provided. The correlation block 572 may be used to correlate data from the device analysis block 568 and therapy usage block 570 to charge session data obtained from a removable memory 580 or, as noted previously via the Internet and a central server. The combination of charging data and therapy data may be used to generate further metrics for the device such as determining an estimate of quiescent and/or self-discharge current draw of the IPG 550, which may be useful to spot possible malfunction or end-of life issues. Charging efficiency and therapy efficiency could also be assessed using various metrics such as identifying how different therapy outputs or types may affect battery current drain to determine if any unexpected or simply inefficient combinations are being used. Such metrics may be informative in particular as the patient is allowed to tailor therapy during use. The correlation block 572 may take as inputs therapy usage data and charge data to generate device data using any of the metrics just described, or using other metrics.

FIG. 11 shows a data structure of an illustrative charge record. A charging record is shown at 600. The charging record may be numbered for a given device or memory device, if desired and as shown. The illustrative charge record may include several bits or bytes of data. For example, the data may include a time stamp for beginning and/or end of the charging session. The identification of the IPG that was charged may be stored, as may be the identification of the charger being used. The charge duration and efficiency during charging may also be stored, as shown. In addition to the data pieces shown, the charge record may include the depth of discharge of the IPG battery at the start and end of charging, the depth of discharge of the charger battery at the start and end of charging, or other useful data, as desired.

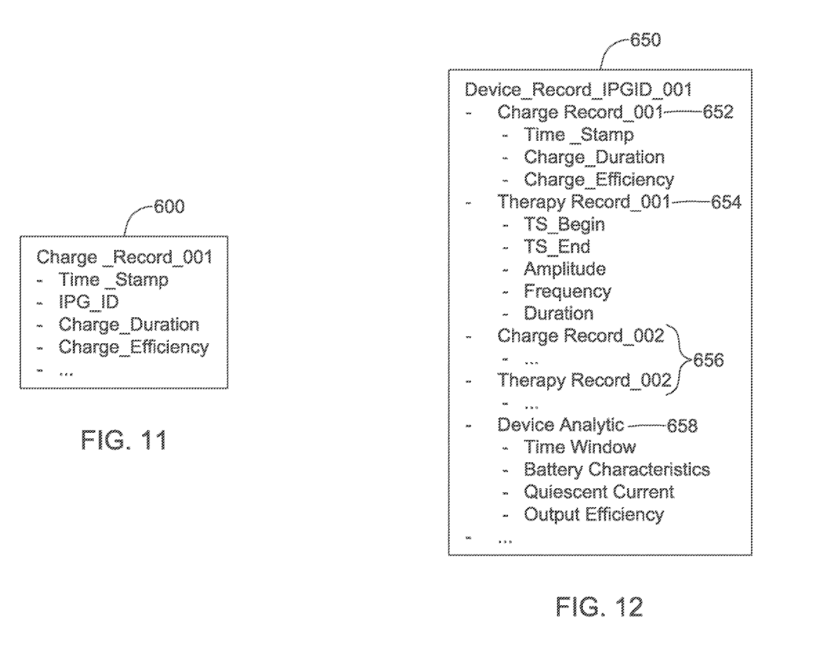

FIG. 12 shows a data structure of an illustrative device record. The device record 650 may contain separately provided charge record 652 and/or therapy record 654, which may be numbered as shown. Multiple charge records and/or therapy records may be included as indicated at 656. Device analytics may be stored as well, using outputs of the correlation block 572 (FIG. 10) if desired. For example, a time window relevant to the IPG may be indicated, with the time window encompassing, for example, one or multiple of each of the charging records and/or therapy records. Analytic metrics such as the battery characteristics during the time window, for example, may be stored (max charge, min charge, estimated self-discharge or internal impedance, if desired). Other estimates of quiescent current in the IPG and/or output efficiency or output impedance may be calculated and stored. Elements 652, 654, 656 may be omitted in some examples, leaving just the device analytics, if desired.

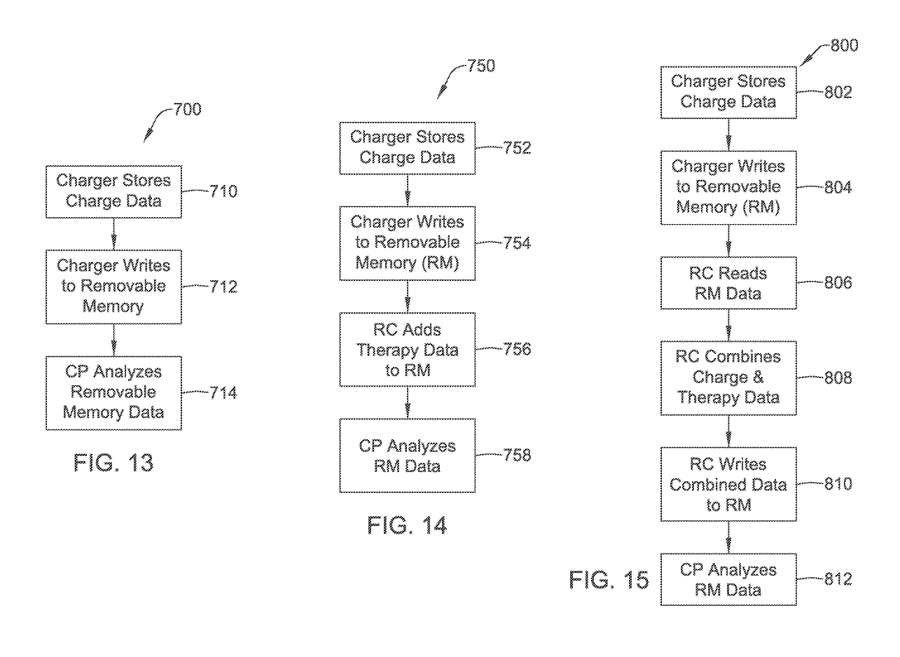

FIGS. 13-15 show illustrative methods in block flow form. Beginning in FIG. 13, a first method is shown at 700. In this method, the charger stores charge data at block 710 during one or more charging sessions. Next, the charger writes the charge data to a removable memory, as shown at 712. The clinician programmer can then be used to analyze the data from the removable memory, as shown at 714. Alternatively, the charger may perform data analysis. In another alternative, the patient remote control may perform analysis. Such analysis may be presented to a physician user at a follow-up visit (which may be in-person or remote).

FIG. 14 shows another method at 750. Here, the charger again stores charge data, as indicated at 752, and writes data to a removable memory (RM) at 754. The patient remote control (RC) then adds therapy data to the RM, as indicated at 756. The patent RC may also provide data related to charge sessions, such as frequency of charging, charging efficiency, begin and end voltage, a relationship between therapy delivered and charging activity, or a warning or alert to let the patient know that the IPG was below or near a battery state where therapy would automatically turn off. Finally, the clinician programmer (CP) analyzes the data on the RM, as shown at 758. As an alternative, the patient remote control (RC) may perform data analysis and may itself be used to present to the clinician. The CP or RC may present information via a user interface such as, for example, frequency of charging, charging efficiency, begin and end voltage, a relationship between therapy delivered and charging activity, or a warning or alert to indicate the IPG was below or near a battery state where therapy would automatically turn off.

FIG. 15 shows another method at 800. Here, the charger stores charge data as shown at 802, and writes that data to a removable memory (RM) at 804. The patient remote control (RC) reads the charging session data from the RM, as indicated at 806. The RC can then combine charge and therapy data at 808, creating one or more of combined records and/or further device analytics as described above. The RC then writes the combined and/or additional data to the RM at 810. The clinician programmer (CP) can then analyze the data from the RM, as indicated at 812. Data can then be presented to the physician by for example, indicating a relationship between therapy delivered and battery usage or charging session activity, indicating how frequently charging occurs, indicating whether a charge session has been initiated in which a device (IPG) battery is below a therapy-off threshold or has even completely discharged, or other data suitable to the system. As an alternative, the patient remote control (RC) may perform data analysis and may itself be used to present to the clinician.

In some embodiments, the removable memory may be provided as a specific thumb drive marked and labeled for the patient to use for the purpose of carrying IPG and related data. Data may be encrypted for storage. A specially formatted removable memory may be provided, such that unauthorized memory devices could be prevented from use in the system, providing security and safety against malware. For example, the clinician programmer and/or patient remote control and/or charger may disable input/output circuitry from reading data files from unauthorized memory devices.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A charging apparatus for use with an implantable medical device, the charging apparatus comprising:
a charging circuit for generating an output magnetic and/or electrical field for charging an implantable medical device;
a monitoring module for determining and storing charging data related to the operation of the charging circuit; and
an output circuit for offloading data from the monitoring module to at least one of a removable memory and/or a separate device connectible wirelessly to the output circuit.

2. The charging apparatus of claim 1 wherein the monitoring module is configured to determine the charging data including at least one of duration of charging events or frequency of charging events without using charge duration or frequency data stored by the implantable medical device.

3. The charging apparatus of claim 1 wherein the monitoring module is configured to send data from the monitoring module over the internet to a central repository.

4. The charging apparatus of claim 1 wherein the monitoring module is configured to store charging records for each of a plurality of charging sessions in which the charging apparatus is used, the charging records comprising:
an identifier for a specific implantable medical device charged in a charging session;
an identifier of when the charging session with the specific implantable medical device occurred; and
at least one parameter of the charging session with the specific implantable medical device.

5. The charging apparatus of claim 4 wherein the at least one parameter includes a measure of charging efficiency obtained by communication with an implantable medical device while charging the implantable medical device.

6. An implantable medical device patient system comprising:
a charging apparatus as in claim 1;
an external control device configured for communication with the implantable medical device and controlling therapy outputs of the implantable medical device;
wherein:
the output circuit of the charging apparatus is configured to load charging data on a removable memory device;
the external control device is configured to read and/or display charging data from the removable memory device.

7. The system of claim 6 wherein the external control device takes the form of a patient remote control or a clinician programmer and the external control device comprises a memory to store therapy usage data for the implantable medical device, a correlation module to correlate charging data from the charging apparatus to the therapy usage data and a device analysis module to determine characteristics of the operation of the implantable medical device from the charging data and the therapy usage data.

8. The system of claim 6, wherein the characteristics of the operation of the implantable medical device comprise an indication of the status of a rechargeable power supply of the implantable medical device.

9. The system of claim 6, wherein the characteristics of the operation of the implantable medical device comprise an indication of the efficiency of charging of the implantable medical device.

10. The system of claim 6 wherein the monitoring module of the charging apparatus is configured to determine the charging data including at least one of duration of charging events or frequency of charging events without using charge duration or frequency data stored by the implantable medical device.

11. The system of claim 6 wherein the monitoring module of the charging apparatus is configured to send data from the monitoring module over the internet to a central repository.

12. The system of claim 6 wherein the monitoring module of the charging apparatus is configured to store charging records for each of a plurality of charging sessions in which the charging apparatus is used, the charging records comprising:
an identifier for a specific implantable medical device charged in a charging session;
an identifier of when the charging session with the specific implantable medical device occurred; and
at least one parameter of the charging session with the specific implantable medical device.

13. The system of claim 6 wherein the charging apparatus is configured such that at least one parameter includes a measure of charging efficiency obtained by communication with an implantable medical device while charging the implantable medical device.

14. An implantable medical device patient system comprising:
a charging apparatus as in claim 1;
a patient remote control configured for communication with the implantable medical device and controlling therapy outputs of the implantable medical device;
wherein:
the output circuit of the charging apparatus is configured to communicate with the patient remote control to offload the charging data from the monitoring module; and
the patient remote control comprises a memory to store therapy usage data for the implantable medical device, a correlation module to correlate charging data from the charging apparatus to the therapy usage data and a device analysis module to determine characteristics of the operation of the implantable medical device from the charging data and the therapy usage data.

15. The system of claim 14, wherein the characteristics of the operation of the implantable medical device comprise an indication of the status of a rechargeable power supply of the implantable medical device.

16. The system of claim 14, wherein the characteristics of the operation of the implantable medical device comprise an indication of the efficiency of therapy delivery of the implantable medical device.

17. The system of claim 14, further comprising a first implantable medical device having in the range of about two to about thirty-two electrical contacts for coupling to an implantable lead, and a rechargeable power supply and therapy circuit therein for delivering therapy outputs via the electrical contacts.

18. The system of claim 17, further comprising at least a second implantable medical devices each having a rechargeable power supply, wherein the charging data comprises records for each of the first and second medical devices.

19. An implantable medical device system comprising:
at least one implantable medical device having about two to about thirty-two electrodes thereon for therapy delivery, therapy circuitry for providing therapy via the electrodes, a rechargeable power source and charging circuitry for charging the rechargeable power source using power received from a charger apparatus;
a charger apparatus comprising:
a charging circuit for generating an output magnetic and/or electrical field for charging an implantable medical device;
a monitoring module for determining and storing charging data related to the operation of the charging circuit; and
an output circuit for offloading charging data from the monitoring module to a removable memory;
an external control device configured to communicate with the implantable medical device and control therapy outputs from the implantable medical device, the external control device having a memory to store therapy usage data for the implantable medical device, and having a correlation module for obtaining charging data from the removable memory and correlating the charging data to therapy usage data to generate an output file containing correlated charging and therapy information.

20. A method comprising:
transcutaneously charging an implantable medical device with a charging apparatus in a charging session, wherein the charging apparatus comprises
a charging circuit for generating an output magnetic and/or electrical field for charging an implantable medical device;
a monitoring module for determining and storing charging data related to the operation of the charging circuit; and
an output circuit for offloading data from the monitoring module to a removable memory;
the charger storing data relating to the charging session on a removable memory; and
removing the removable memory and placing it into one of a patient remote control or a clinician programmer to obtain charge data therefrom; and
the clinician programmer or the patient remote control presenting data relating to the charge data to a user thereof.
